(19) United States
(12) Patent Application Publication
(10) Pub. No.: US 2017/0290639 A1
(43) Pub. Date: Oct. 12, 2017

(54) TETHER FOR INTRAORAL DEVICES

(71) Applicant: Epoch Dental LLC, Morton Grove, IL (US)

(72) Inventor: Louis Kaufman, Morton Grove, IL (US)

(73) Assignee: Epoch Dental LLC

(21) Appl. No.: 15/095,913

(22) Filed: Apr. 11, 2016

(51) Int. Cl. A61C 5/02; A61C 15/04; A61B 90/57; A61C 19/00

(52) U.S. Cl. CPC A61C 5/028; A61C 19/00; A61C 15/041; A61B 90/57

(57) ABSTRACT

A safety device for tethering an object during intraoral use, such as a dental finger driver. A tether includes a semi-rigid tip portion connected to a flexible, inelastic tail. The tip portion is oriented parallel with the length of the tail during use. The tail may be formed from a filament, such as nylon monofilament. The tip portion may be formed from plastic. The tip can be easily inserted through an aperture in the device being tethered, with the tail portion being pulled through the aperture and secured to the intraoral object. The tail portion may be secured by a knot, a stopper, or a loop formed in a second end of the tail, through which the tip may be passed.

### TECHNICAL FIELD

The present disclosure relates to the securing of intraoral devices during dental operations. More specifically, this disclosure relates to an apparatus and method of relatively quickly securing a tether to an intraoral device to mitigate the effects of accidental release of said device down the throat of a person leading to choking and other complications, or down to the floor leading to a loss of sterility.

### BACKGROUND

When one visits the dentist, it is expected that some of that time will be spent in a reclined position. The reclined position is advantageous as it allows dental professionals optimal access to all parts of the mouth. However, this position has the disadvantage that if any small object drops into the mouth, the effects of gravity promote travel of the object down the patient's throat.

Although very uncommon, when an object is accidentally released over a patient's mouth, the consequences of ingestion or aspiration can be serious. In addition to panic, gagging, and choking, ingestion or aspiration of small objects can lead to surgery to remove the object. Patients can suffer, and resulting liability can be very costly for the dental professional.

In response, dental professionals have tried many methods to mitigate the risk of objects falling down a patient's mouth. Some methods include: changing the position of the patient, use of gauze, use of dental dams, and use of floss as a tether.

One method used to reduce that chance that small objects will fall down a patient's throat is to have the patient positioned in a more upright position than the normal relined and supine position. A more upright position decreases the angle of the mouth opening relative to the ground. As a result, as the angle is decreased, objects that fall in the mouth will face increase contact and resistance from the mouth and tongue and the chance of aspiration or ingestion is decreased. However, as the patient is placed in a more upright position, illumination of the inside of the mouth becomes more challenging and the dental professional's access to the mouth is hindered, negatively impacting the comfort and efficacy of the dental professional.

Another method involves the use of gauze placed in the back of the mouth of a patient. Although this creates a barrier for objects from entering the throat, it may cause patient discomfort by hindering normal breathing, drying out the mouth, and causing a sense of claustrophobia and general irritation in the mouth. In addition, gauze in the mouth may temporarily hinder the dental professional's access to parts of the mouth being worked on.

Some doctors will use dental dams to reduce drops into the throat. Dental dams are held over individual teeth or groups of teeth and are placed in a way that blocks the path to the throat. However, as the dental dam covers the mouth, patients are forced to breathe through their nose which can be uncomfortable if the patient has a cold. Also, the ability for the patient to communicate with the dental professional is impaired. Dental dams can also be costly and take a significant amount of time to deploy.

In light of the many disadvantages to previously-described techniques for reducing risk of dropped objects, some dental professionals use floss to restrict the motion of objects. For example, one common tool used in dental procedures is an implant finger driver, which is a small tool designed to be rotated by a dentist within the patient's mouth. To reduce harm from accidental dropping of finger drivers down a patient's throat, some of these finger drivers have been designed to include a safety aperture or eyelet to allow a piece of dental floss to be threaded through and tied as a tether.

If the tool is dropped down a patient's throat, the floss can be used to limit the tool's travel and/or retrieve the tool. In addition, floss is relatively inexpensive, disposable and sterile. However, threading of dental floss through the small aperture of the tool can be very challenging, particularly for a dental professional handling the floss with hands in protective gloves. The time and frustration involved in threading floss through a tool safety eyelet, and then tying off the floss, can encourage some professionals to forego tethering of tools during procedures, thereby putting the patient at risk of swallowing or aspirating a dropped tool.

Another tether structure is illustrated in U.S. Patent Application Publication 2011/0132944A1. However, this device includes a complex assembly with numerous metal components proximate the intraoral tool being secured, thereby potentially interfering with the dental professional's handling of the tool, while increasing cost and, if not disposed of between patients, requiring time-consuming sterilization procedures. Yet other tethers include features that must be specially adapted to match the tool being secured, thereby limiting their applications and inhibiting use with a dental professional's existing tools. Examples of tethers requiring special adaptations include those of German Patent Publication DE 20 2010 002 860 U1 and U.S. Pat. No. 4,643,674.

### SUMMARY

One aspect of the present disclosure is directed to a safety device that can be rapidly and easily attached to an intraoral device. The safety device is comprised of a tail portion formed from dental floss or a similarly structured filament. The tail portion is attached to a semi-rigid tip. The tip is used to thread the line through a small aperture formed in a dental finger driver, or other device being used intraorally. In some embodiments, use of a semi-rigid tip permits easy maneuvering of the tip through a small aperture by a dental professional with gloved hands, while also enabling passage of the tip through a curved aperture or an aperture with reduced clearance on its exit side. Preferably, the tip is pointed, to further increase the ease and speed with which the tip may be inserted.

In some embodiments, the tip can be formed from plastic; and the filament can be formed from nylon or plastic. The tip can be attached to the filament via mechanisms including laser welding, chemical welding, heat welding, adhesive or other connection techniques.

In another embodiment, the filament includes a small integrated, pre-formed loop at its end distal to the semi-rigid tip, through which the semi-rigid tip can be passed after threading through a tool's aperture, thereby rapidly securing the tether to the tool and freeing a hand from secure the attachment of the safety.

Preferably, the safety lines are packaged in a sterile manner and disposable after each patient's use, thereby avoiding risk of infection or disease.

In accordance with another aspect, the filament may include a stopper portion attached to the end distal to the semi-rigid tip. The stopper is sized at least slightly larger than the tool aperture through which the filament is to be threaded. The tool can be secured after the semi-rigid tip is passed through the aperture and the filament is pulled through until the stopper contacts the aperture periphery, thereby eliminating the need to tie the filament to the tool. Various methods for securing an intraoral device are also provided.

These and other embodiments will become apparent in light of the disclosure elsewhere herein.

### BRIEF DESCRIPTION OF THE FIGURES

FIG. 1 is a perspective view of a tether, in accordance with one embodiment.

FIG. 2 is a perspective view of a prior art finger driver with safety tether aperture.

FIG. 3 is a perspective view of a tether having a stopper portion.

FIG. 4 is a perspective view of a tether having a loop termination.

FIG. 5 is a perspective view of the tether of FIG. 4, part way through attachment to a finger driver.

FIG. 6 is a perspective view of the tether of FIG. 4, attached to a finger driver.

FIG. 7 is a perspective view of a tether, in accordance with another embodiment.

FIG. 8 is a perspective view of the tether of FIG. 7, part way through attachment to a finger driver.

FIG. 9 is a front elevational view of a tether clamp.

FIG. 10 is a side view of a tether clamp.

### DESCRIPTION OF THE PREFERRED EMBODIMENTS

While this invention is susceptible to embodiment in many different forms, there are shown in the drawings and will be described in detail herein several specific embodiments, with the understanding that the present disclosure is to be considered as an exemplification of the principles of the invention to enable any person skilled in the art to make and use the invention, and is not intended to limit the invention to the embodiments illustrated.

In accordance with some embodiments, an improved tether can be used with finger drivers and other intraoral devices. The tether is adapted for rapid and easy deployment by individuals wearing safety gloves, thereby encouraging regular use. Preferably, the tether can be produced inexpensively and used as a disposable product, thereby maintaining a sterile operatory environment without time consuming post-operatory sterilization.

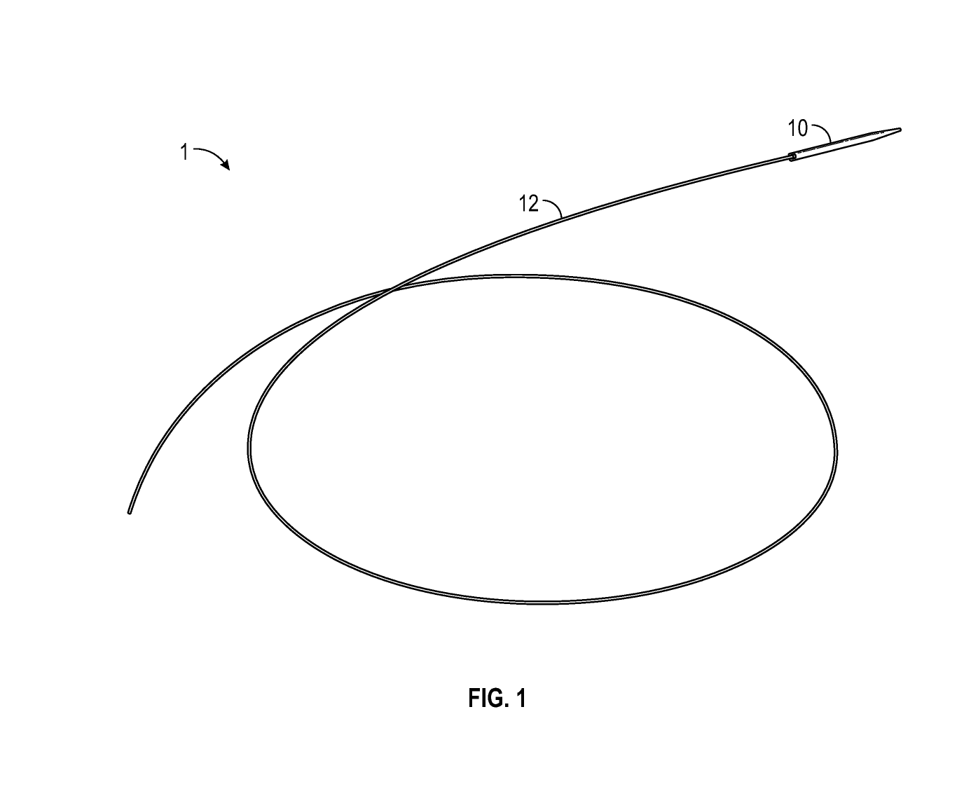

FIG. 1 illustrates a safety tether 1 in accordance with one embodiment. The tether 1 includes a tip portion 10 and a tail portion 12. In some embodiments, tip 10 may be approximately one inch in length. Tip 10 may have a body that is cylindrical in cross-section, relatively uniform in diameter for most of its length, and pointed at a distal end. In other embodiments, tip 10 may have other shapes, such as a relatively flat cross-section. The diameter (or widest point, depending on its shape) of tip 10 is adapted for ready insertion into, and passage through, an aperture or eyelet formed in a tool to be secured. For example, in a tether used to secure a finger driver having a safety aperture that is 1.5 mm in diameter, it may be desirable to utilize a tether in which tip 10 has a maximum diameter of 0.75 mm.

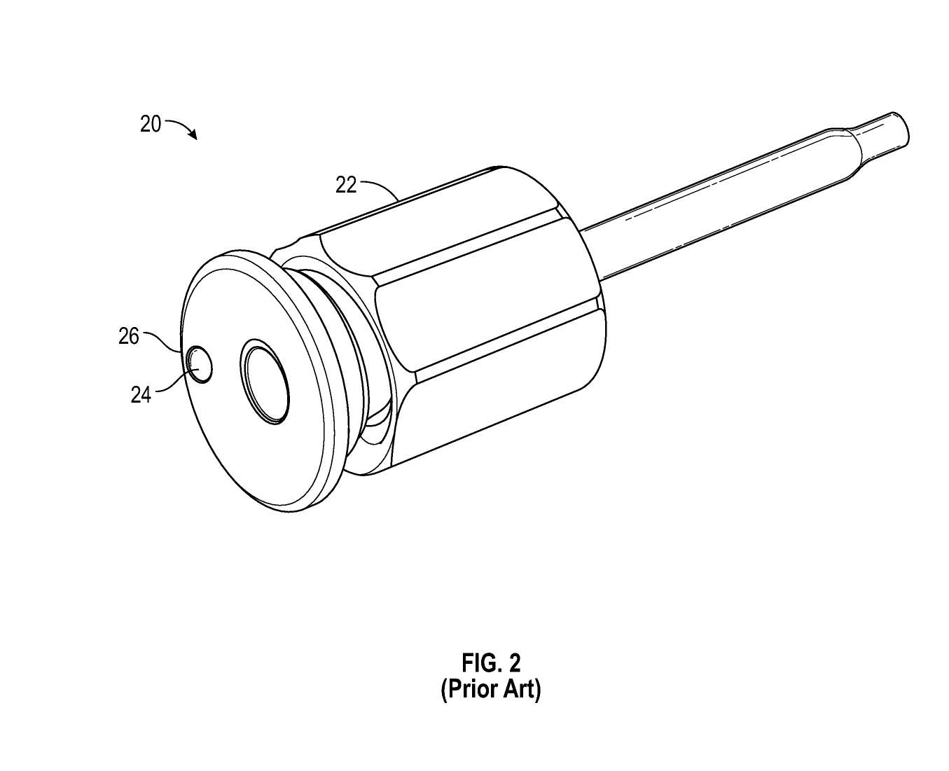

FIG. 2 illustrates a conventional dental finger driver 20 with which tether 1 may be used. Finger driver 20 includes body portion 22. Safety aperture 24 extends through the body portion 22 of finger driver 20.

The construction of tether 1 is specially adapted for rapid and easy deployment. Tip 10 is preferably formed from an elastic material providing moderate levels of rigidity. Providing a semi-rigid tip 10 aids a dental professional in guiding tip 10 into a small aperture—particularly when handled by a gloved hand, and as compared to guidance of conventional (flexible i.e. non-rigid) dental floss. However, avoiding materials that are highly rigid (such as stainless steel) can mitigate the risk of inadvertent puncture of a surgical glove, or injury to a patient or dental practitioner. Also, some tools that are originally manufactured for tethering using conventional dental floss may have a tether aperture that is not a straight path (e.g. an aperture may enter a finger driver end portion, curve within the driver body, and exit through the driver side wall). A highly-rigid structure, such as a conventional needle, may not be able to thread through such a curved aperture, while semi-rigid tip 10 can pass easily therethrough.

A proximal end of tip 10 is connected to tail 12. Tail 12 is a long, thin filament or line that can be used to secure a dental tool or other intraoral object to an object outside the patient's mouth, such as the dental professional, the patient or an object in the operatory environment. Tip 10 and tail 12 are oriented parallel to one another, and preferably share a common lengthwise axis. Tail 12 may be formed from a highly flexible, inelastic material so that it does not significantly interfere with movement of the tool or object to which it is attached during use. Tail 12 will preferably have good tensile strength, so that it may be used to retrieve a dental tool without breaking. For example, tail 12 may be formed from dental floss, monofilament nylon line, or braided and/or thermally fused multifilament lines.

In accordance with one exemplary embodiment, tip 10 is formed from semi-rigid, flexible plastic, while tail 12 is formed from monofilament nylon line. Tip 10 can be fused to tail 12 via thermal welding, such as a laser weld, to quickly and inexpensively form an integrated, inexpensive, disposable assembly having a semi-rigid portion and a flexible portion. In other embodiments, tip 10 can be connected to tail 12 via other means, such as chemical welding, crimp connection or adhesives. Tail 12 is preferably at least 12 inches in length, and most preferably at least approximately 20 inches in length.

In operation, tip 10 can be readily guided through aperture 24, and then pulled to thread portions of tail 12 through aperture 24. Tail 12 can then be tied off around edge portion 26 of tool 20. Another portion of tail 12 can be secured to something external to the patient's mouth. In the event that tool 20 is accidentally ingested or aspirated, tail 12 can then be used to pull tool 20 from a patient's throat.

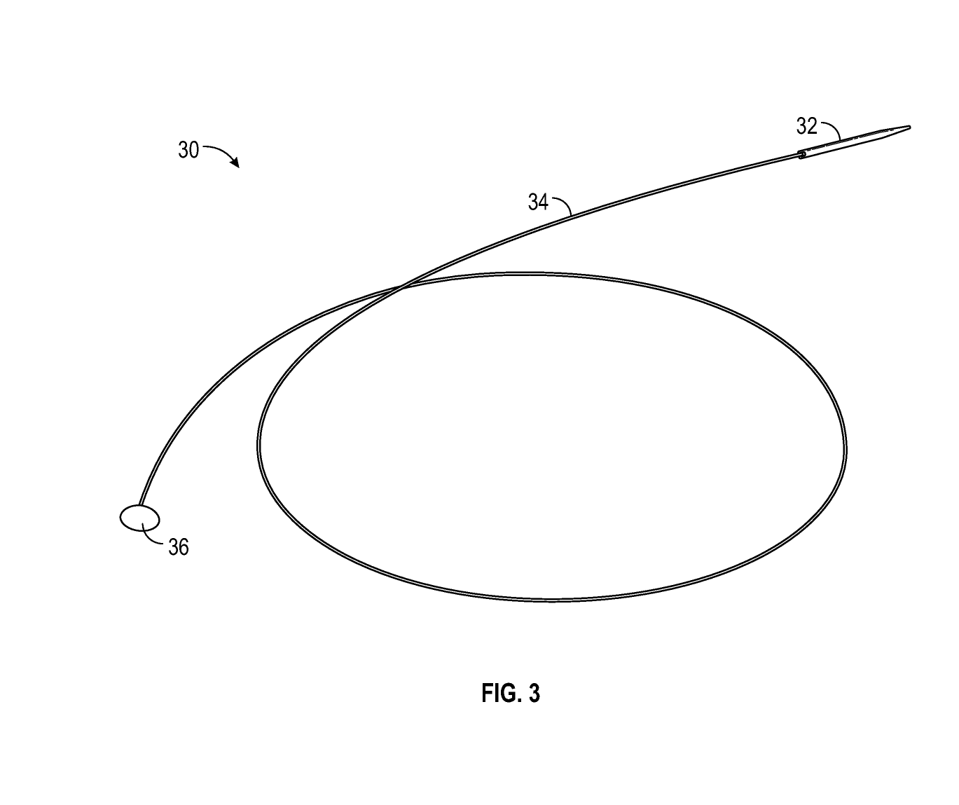

While the embodiment of FIG. 1 may assist in passing the tether through a tool safety aperture, time and manual dexterity may still be required to tie off the tether to the tool and/or a securing point outside the patient's mouth. Therefore, in some embodiments, the tether may include additional features to facilitate securing of the tether to the tool, potentially including one-handed attachment of the tether. FIG. 3 illustrates such an embodiment. Tether 30 includes tip portion 32 and tail portion 34, which are constructed similarly to analogous portions of tether 1 in FIG. 1. Tether 30 further includes stopper portion 36, affixed to an end of tail 34 opposite that of tip 32. Stopper 36 has a diameter that exceeds the diameter of tail 34, as well as the diameter of a tool tether aperture through which tail 34 passes. In operation, tip 32 can be used to readily thread tether 30 through a tool aperture (e.g. aperture 24 in tool 20). The user can then continue to feed tether 30 through the tool aperture, until stopper 36 engages with the periphery of the tool aperture (e.g. tool portion 26 of tool 20), thereby securing tether 30 to the tool. The portion of tail 34 that is proximate tip 32 can then be tied off or otherwise secured to a location outside the patient's mouth.

Stopper 36 can be formed in a variety of ways. In one embodiment, stopper 36 can be formed by tying tail 34 into a knot at one end, or wrapping one end of tail 34 upon itself and forming stopper 36 by thermally fusing the segment. In both cases. a segment of tail 34 is used to provide a larger-diameter stopper component without requiring any additional tether materials. In another embodiment, stopper 36 can be formed from an additional component that is secured to the end of tail 34 that is opposite tip 32. For example, where tail 34 is formed from nylon monofilament, stopper 36 can be formed from a small nylon plug thermally fused to and end of tail 34.

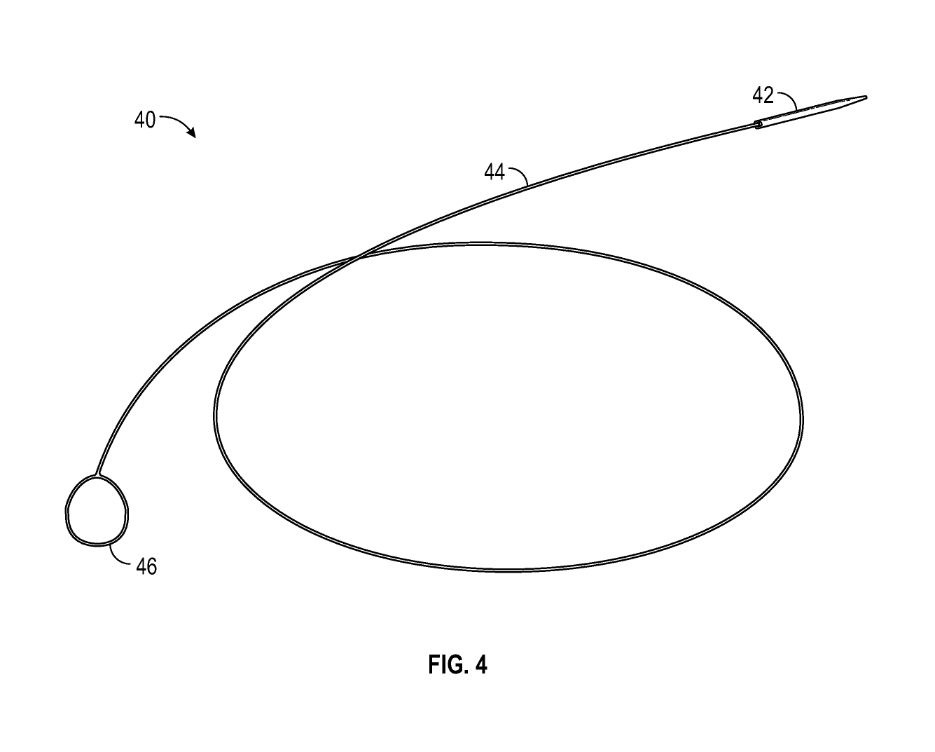

While the embodiment of FIG. 3 enables a user to rapidly secure tether 30 to an intraoral device, some users may feel that the additional size of stopper 36, butted up against a surface of an intraoral tool, impedes a dental professional's dexterity or comfort while handling the tool. FIG. 4 illustrates another tether embodiment, facilitating rapid attachment to a tool, while also minimizing impact on tool handling. Tether 40 is formed from tip 42 and tail 44, which are similar in construction to analogous components of tether 30 in FIG. 3. However, tether 40 further includes loop 46 formed at an end of tail 44 opposite that of tip 42.

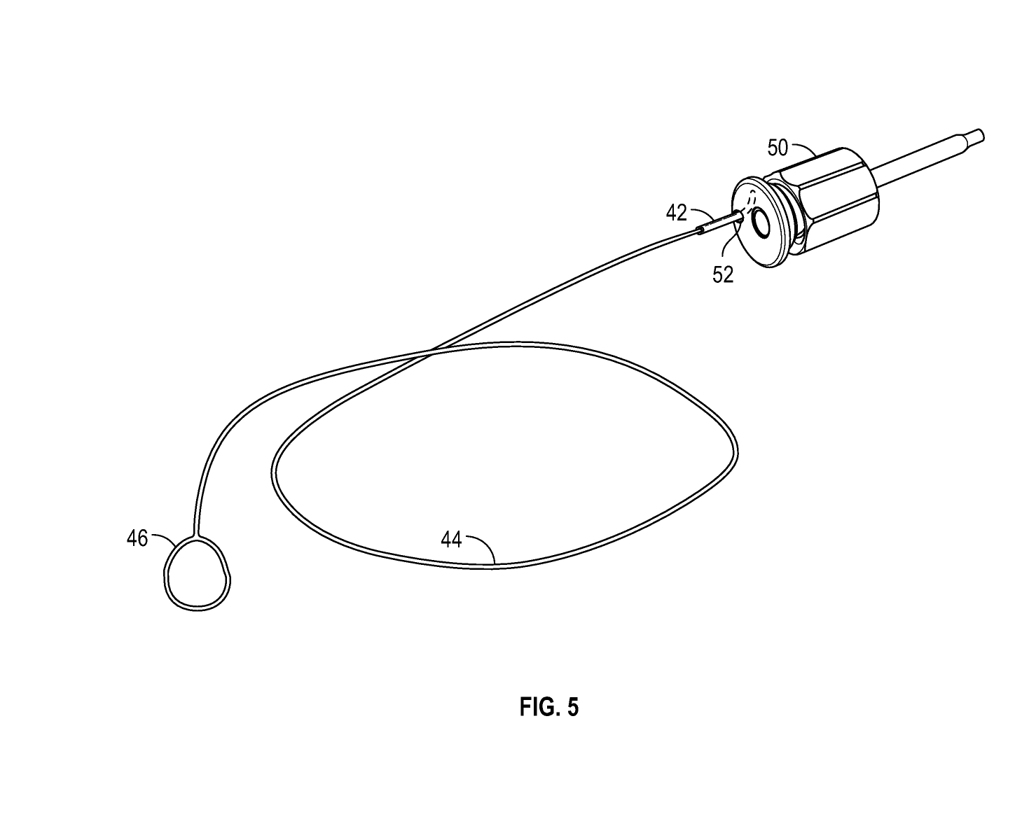

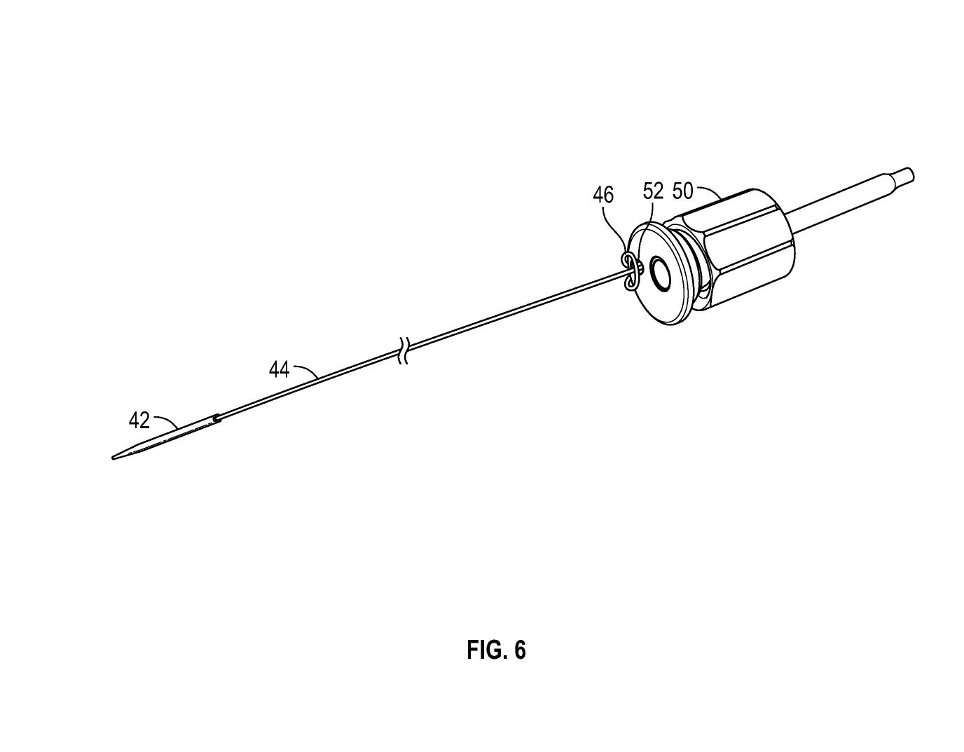

FIG. 5 illustrates tether 40 in mid-deployment. Tip 42 is inserted through aperture 52 in finger driver 50. Tip 42 and a portion of tail 44 are subsequently pulled through aperture 52, and fed back through the center of loop portion 46. A user continues to pull tip 42 and tail 44 through loop 46, until the line wraps tightly around a perimeter of aperture 52. FIG. 6 illustrates tether 40, after being secured to finger driver 50. While tether 40 can be deployed rapidly and consistently, when secured to an intraoral device, only relatively fine tether line (such as monofilament line) is proximate the device, thereby minimizing any impact on comfort or handling by the dental professional.

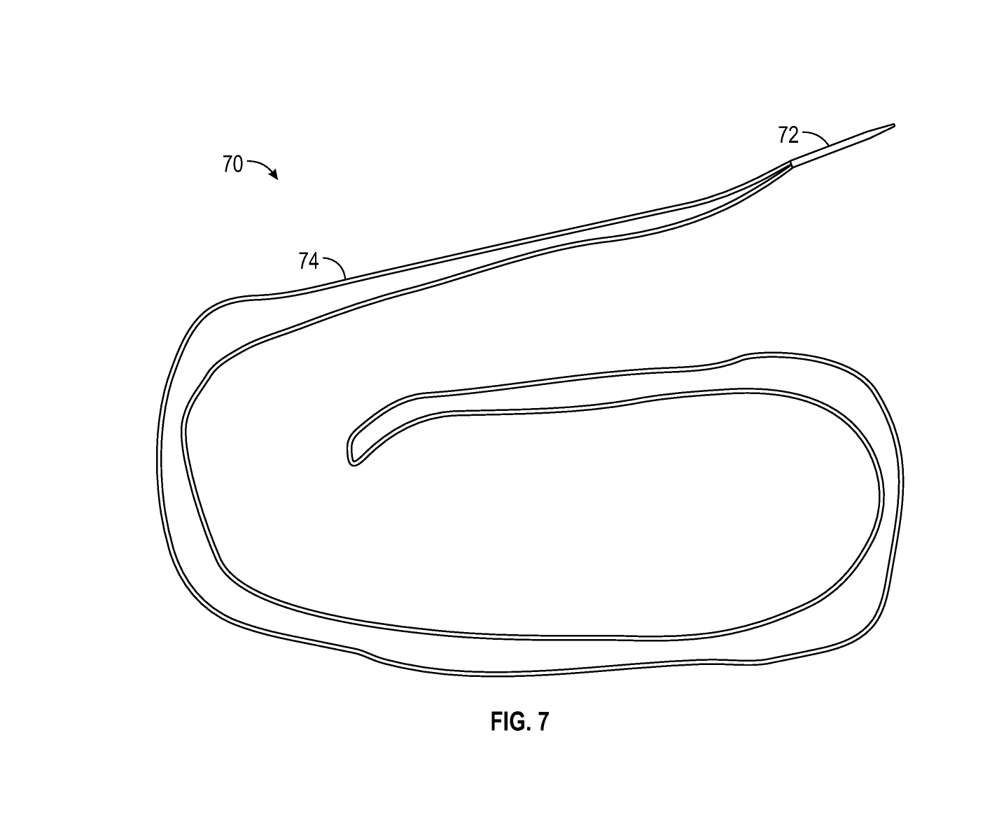

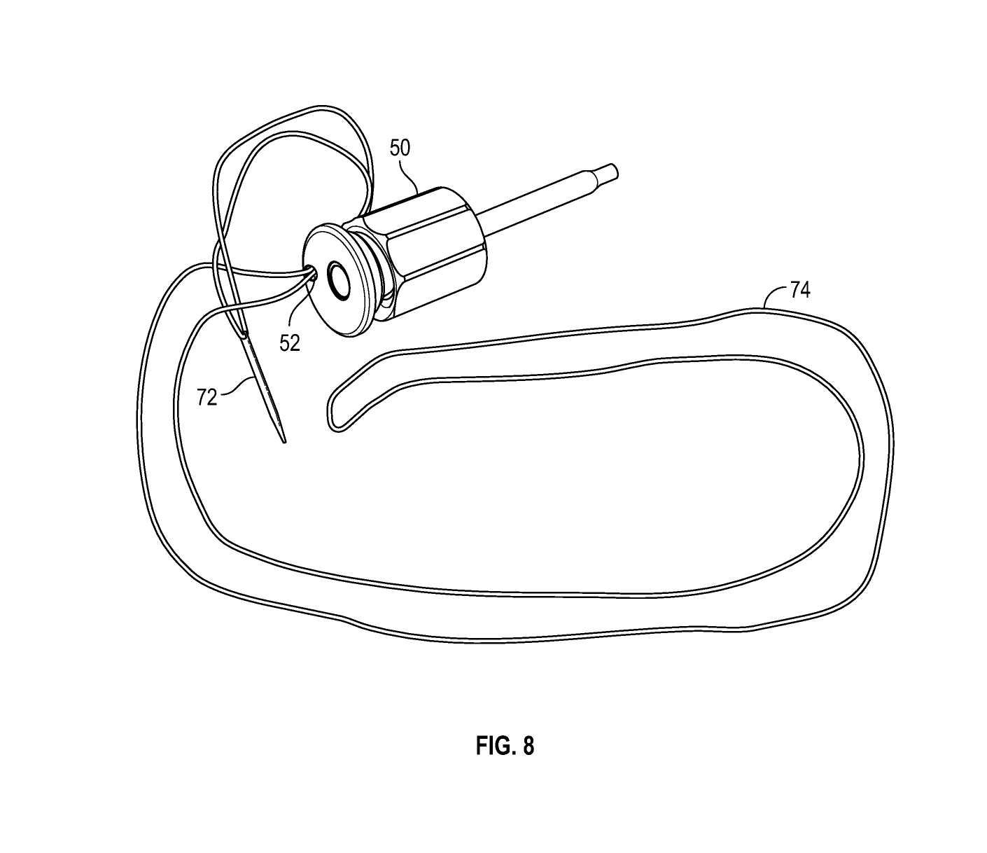

In yet another embodiment, illustrated in FIG. 7, a tether 70 can be formed from a tail 74 comprised of a single loop of line material, attached to tip portion 72. Such an embodiment facilitates passage of tip 72 through (large) tail loop 74, after passing through a tool aperture. FIG. 8 illustrates tether 70, with tip 72 passed through aperture 52 of tool 50, and fed back through the loop formed by tail 74. When pulled tight, tether 70 provides dual, parallel lengths of line from tail portion 74, potentially providing increased tensile strength of tail 74 for reliable tool retrieval. However, because tail 74 is preferably formed from a very thin filament, the parallel lengths of tail 74 may have little negative impact on the handling of tool 50 to which it is secured.

Embodiments of the tethers described above can be preferably formed from materials that are inexpensive and easy to manufacture. They can therefore be utilized in a disposable manner. In order to maintain a sterile operatory environment, the tethers may be manufactured in a sterile environment and then separately packaged in sterile packaging. A dental professional can then open a new package each time a tether is required, with the tether being disposed of after each use.

As described above, once the tether is attached to an intraoral device, the tether is often further secured to a location outside the patient's mouth. For example, the tether can be tied off into a dental professional's finger or wrist, tied to an article worn by the dental professional, tied off onto the patient of the patient's clothing, or secured to an object or piece of equipment within the operatory environment. In some operating environments, patients are wearing bibs secured by clips such as alligator clips; in such circumstances, the tether may be secured by a bib clip.

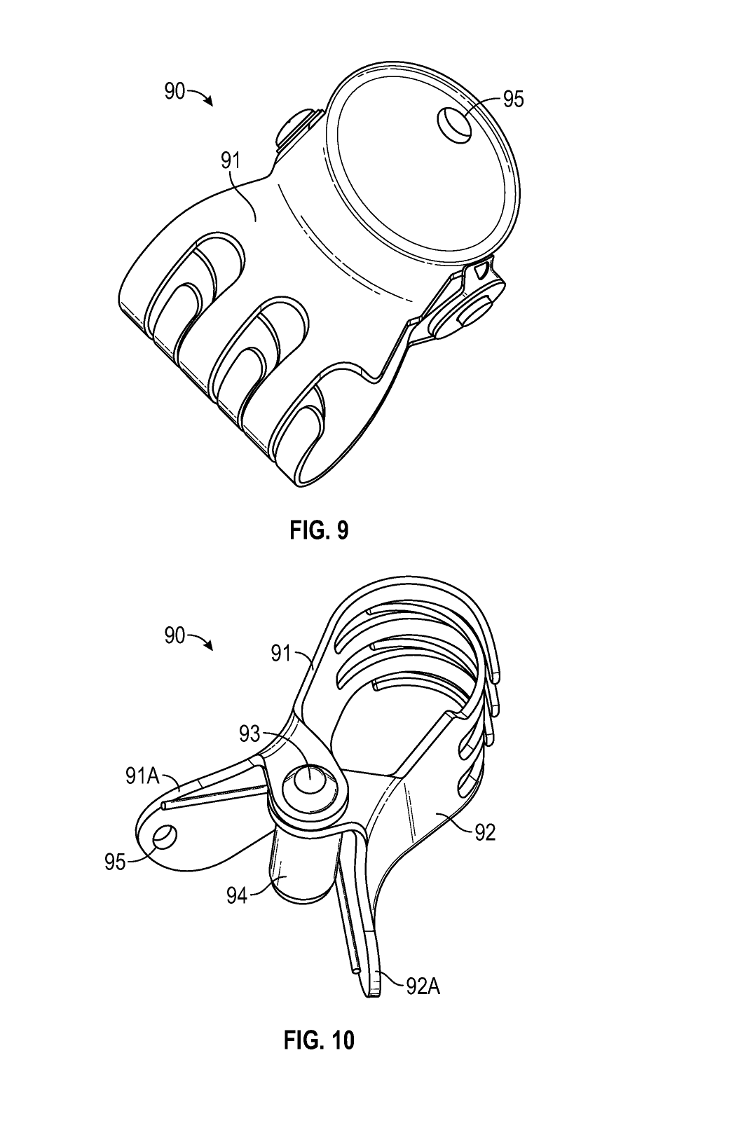

In accordance with another aspect of the embodiments, a clip may be provided to further facilitate attachment of the tether outside the patient's mouth. FIGS. 9 and 10 illustrate such an embodiment. Clip 90 is spring-loaded and preferably formed from stainless steel. Claws 91 and 92 are attached at hinge 93, and biased against one another by coil spring 94. Aperture 95 is provided in a portion of claw 91. Rear portions 91A and 92A of claws 91 and 92 can be squeezed by a dental professional in order to open claws 91 and 92; the clamp can then be released to attach the clamp to locations such as the dental professional's finger, shirt cuff or collar, the patient's clothing, an equipment tray or another nearby object in the operatory environment. Meanwhile, the tip portion of a tether embodiment can be readily passed through aperture 95 and tied off on clip 90.

In other embodiments, clip 90 can be adapted for use as a bib clip. Specifically, a spring-actuated bib clip may be provided having a tether aperture such as aperture 95 in clip 90, such that the same clip utilized for securing a patient's bib can also be readily utilized to anchor a safety tether.

While embodiments are illustrated in use with a finger driver tool, it is contemplated and understood that the tethers described herein may be beneficially utilized to secure a variety of other devices and objects as well, particularly to the extent such objects include apertures through which a tether tip portion may be passed. For example, in dental applications, the tethers may be used to secure items such as implant guide pins and/or dental appliances.

The previous description of the disclosed embodiments is provided to enable any person skilled in the art to make or use the invention disclosed herein. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein. All references cited herein are expressly incorporated by reference.

What is claimed is:

1. A safety device for tethering tools or other objects utilized intraorally and having an aperture formed therein, the device comprising:
a tail portion comprised of a length of relatively non-rigid and non-elastic filament; and
a semi-rigid elastic tip connected to a first end of the tail portion, the tip being oriented in parallel with the first end of the tail portion to which it is attached.

2. The safety device of claim 1, further comprising a loop formed at a second end of the tail portion, through which the tip can be passed.

3. The safety device of claim 1, in which the tail portion filament forms a large loop.

4. The safety device of claim 1, wherein the tail portion is comprised of dental floss.

5. The safety device of claim 1, wherein the tail portion is comprised of nylon.

6. The safety device of claim 5, wherein the tail portion is comprised of nylon monofilament.

7. The safety device of claim 1, wherein when the tail portion when pulled taut is at least approximately 20 inches in length.

8. The safety device of claim 1, in which the tip is formed from plastic.

9. The safety device of claim 8, in which the tail portion is formed from nylon filament; and in which the tail portion is connected to the tip via thermal welding.

10. The safety device of claim 1, further comprising a stopper portion connected to a second end of the tail portion opposite the first end.

11. The safety device of claim 10, in which the stopper portion is comprised of a knot formed in the tail portion filament.

12. The safety device of claim 10, in which the stopper portion is comprised of a portion of the second end of the tail, wrapped upon itself and thermally fused together.

13. The safety device of claim 10, in which the stopper portion is comprised of a plug secured to the second end of the tail portion filament.

14. The safety device of claim 1, further comprising a clip for attachment to a location outside of a patient's oral cavity, the clip including an aperture through which the tip may be passed for securing the tail portion to the clip.

15. A method of quickly and temporarily tethering a safety line to an intraoral device, said method comprising:
gripping onto a safety line comprised of a tail portion formed from a length of relatively non-rigid and non-elastic filament, and a semi-rigid elastic tip connected to a first end of the length of filament in a configuration parallel with said first end;
threading the semi-rigid elastic tip through an aperture formed in an intraoral device; and
securing a second end of the length of filament to the intraoral device.

16. The method of claim 15, in which the step of securing a second end of the length of filament to the intraoral device comprises: tying a knot proximate the second end of the length of filament.

17. The method of claim 15, in which the step of securing a second end of the length of filament to the intraoral device comprises: feeding the tip through a loop formed in the second end of the length of filament; and pulling the length of filament tight against the intraoral device.

18. The method of claim 15, further comprising: attaching the first end of the length of filament to a location external to the patient's oral cavity.

19. The method of claim 18, wherein the step of attaching the first end of the length of filament to a location external to the patient's oral cavity comprises: attaching a clip having an aperture therein to a location external to the patient's oral cavity; passing the tip through the clip aperture; and tying a portion of the filament proximate the first end to the clip.
